(19) United States
(12) Patent Application Publication
(10) Pub. No.: US 2026/0315290 A1
(43) Pub. Date: Oct. 15, 2026

(54) Absorbent Pad Assembly

(71) Applicant: Sarah Mutairayo, West Boylston, MA (US)

(72) Inventor: Sarah Mutairayo, West Boylston, MA (US)

(21) Appl. No.: 19/178,106

(22) Filed: Apr. 14, 2025

(51) Int. Cl. A61F 13/472; A61F 13/56

(52) U.S. Cl. CPC A61F 13/47236; A61F 13/5616

(57) ABSTRACT

An absorbent pad assembly for absorbing bodily fluids includes a pad that has a plurality of intersecting sides such that the pad has a wedge shape. In this way the pad can be positioned between a user's buttocks. The pad is comprised of a fluid absorbent material to absorb bodily fluids. A panel is bonded to the pad and the panel extends outwardly beyond either side of the pad to define a pair of wings each extending away the pad. A pair of adhesive members is each bonded to a respective one of the wings defined on the panel to adhere to the user's skin for retaining the pad between the user's buttocks.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to absorbent device and more particularly pertains to a new absorbent device for absorbing bodily fluids. The device includes a wedge shaped pad that is positionable between a user's buttocks and a pair of adhesive pads that are disposed on the wedge shaped pad. The adhesive pads adhere to the user's skin to retain the pad in a preferred orientation for absorbing bodily fluids that leak from a feminine hygiene pad.

### (2) Description of Related Art Including Information Disclosed Under 37 CFR 1.97 and 1.98

The prior art relates to absorbent devices including a variety of bodily fluid pads that each has a generally elongate tubular shape for positioning between a user's buttocks for absorbing bodily fluids. The prior art discloses a bodily fluid pad which includes an absorbent pad and a pair of wings that each engage a respective user's buttocks for retaining the absorbent pad between the user's buttocks. The prior art discloses a variety of feminine hygiene pads which each at least includes a rectangular pad portion and a tail portion extending away from the pad portion such that the tail portion is positionable between a user's buttocks.

### (h) BRIEF SUMMARY OF THE INVENTION

An embodiment of the disclosure meets the needs presented above by generally comprising a pad that has a plurality of intersecting sides such that the pad has a wedge shape. In this way the pad can be positioned between a user's buttocks. The pad is comprised of a fluid absorbent material to absorb bodily fluids. A panel is bonded to the pad and the panel extends outwardly beyond either side of the pad to define a pair of wings each extending away the pad. A pair of adhesive members is each bonded to a respective one of the wings defined on the panel to adhere to the user's skin for retaining the pad between the user's buttocks.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an absorbent pad assembly according to an embodiment of the disclosure.

FIG. 2 is a front view of an embodiment of the disclosure.

FIG. 3 is a back view of an embodiment of the disclosure.

FIG. 4 is a top view of an embodiment of the disclosure.

FIG. 5 is a right side view of an embodiment of the disclosure.

FIG. 6 is a perspective in-use view of an embodiment of the disclosure.

FIG. 7 is a perspective view of an alternative embodiment of the disclosure.

### (j) DETAILED DESCRIPTION OF THE INVENTION

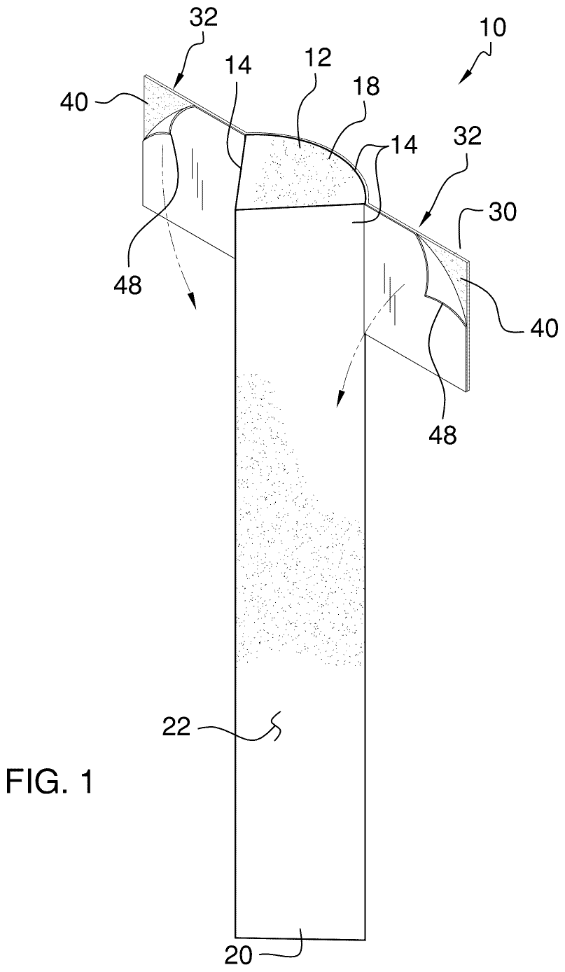

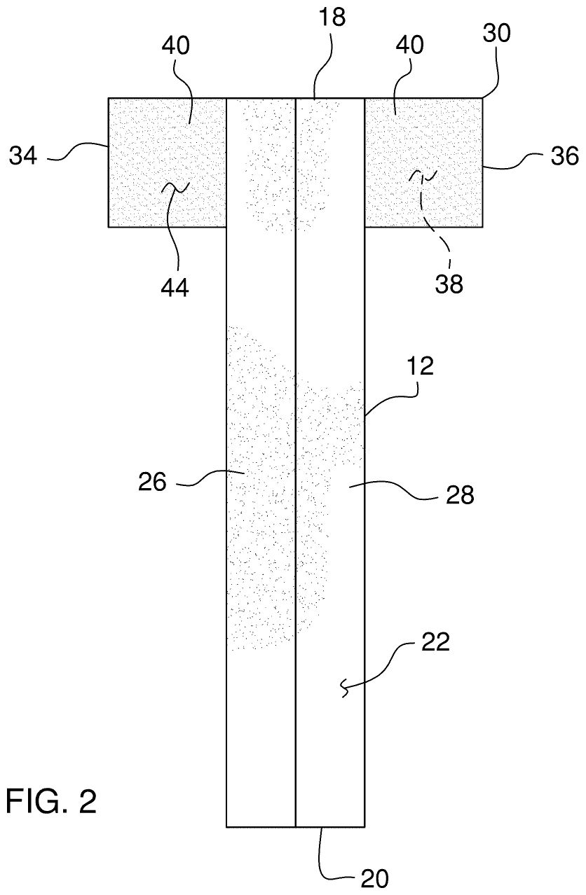

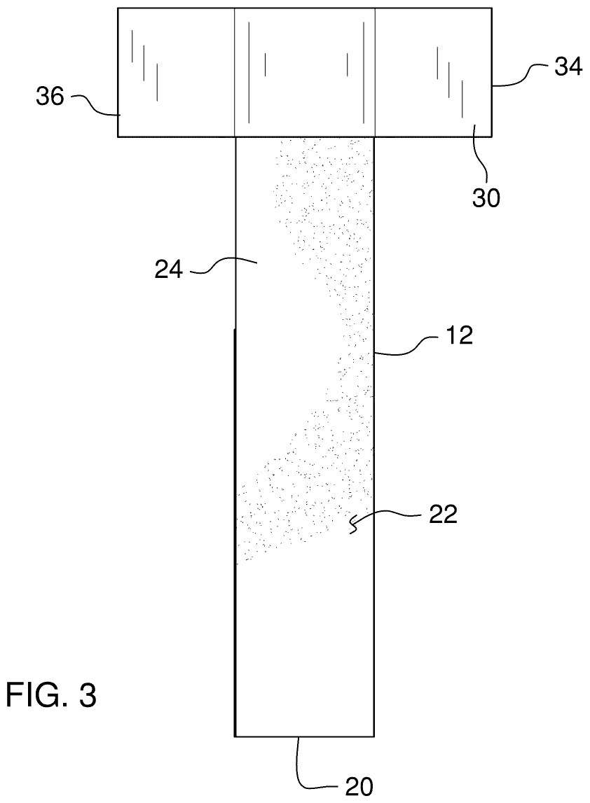

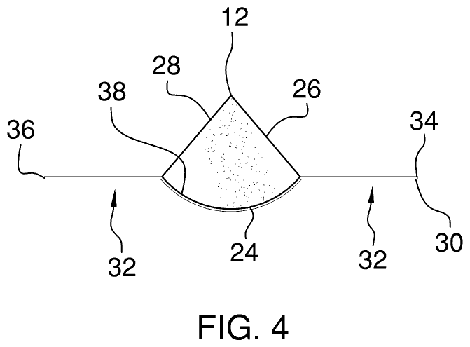

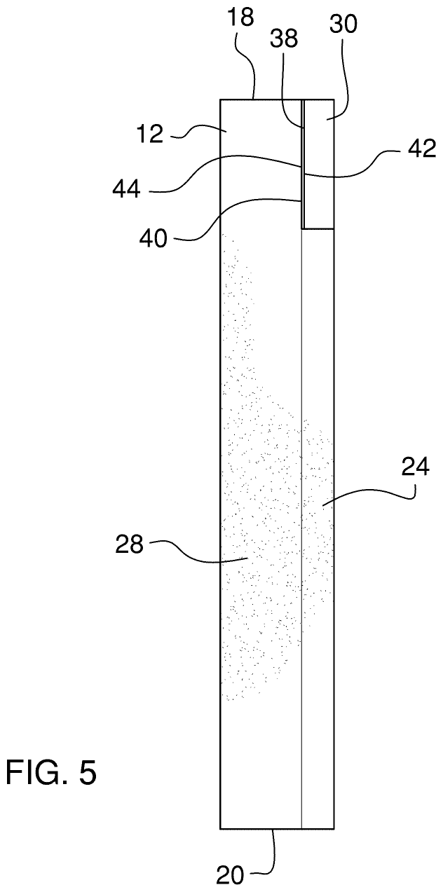

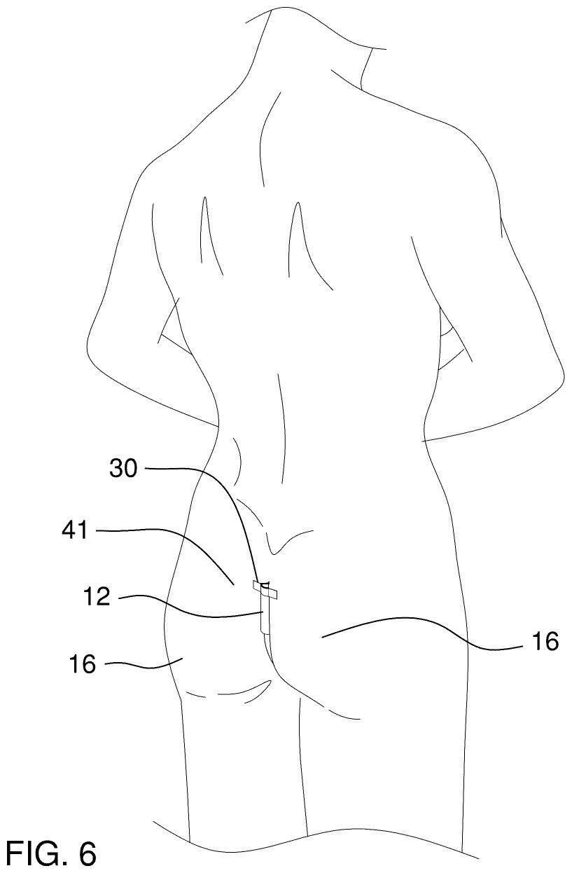

With reference now to the drawings, and in particular to FIGS. 1 through 7 thereof, a new absorbent device embodying the principles and concepts of an embodiment of the disclosure and generally designated by the reference numeral 10 will be described.

As best illustrated in FIGS. 1 through 7, the absorbent pad assembly 10 generally comprises a pad 12 that has a plurality of intersecting sides 14 such that the pad 12 has a wedge shape. In this way the pad 12 can be positioned between a user's buttocks 16. The user may be a female that is menstruating and who is employing a feminine hygiene pad for absorbing menstrual fluids. The pad 12 is comprised of a fluid absorbent material such that the pad 12 can absorb bodily fluids. In this way the pad 12 can absorb menstrual fluids that commonly leak from a feminine hygiene pad when a female is lying on her back in a prone position. Thus, the pad 12 inhibits clothing from becoming accidentally stained with leaked menstrual fluids or other bodily fluids that otherwise might soil the clothing.

The pad 12 has a first end 18, a second end 20 and an outer surface 22 extending between the first end 18 and the second end 20. The pad 12 is elongated between the first end 18 and the second end 20, and each of the intersecting sides 14 of the pad 12 is associated with the outer surface 22. The plurality of intersecting sides 14 includes a back side 24, a first lateral side 26 and a second lateral side 28. Each of the first lateral side 26 and the second lateral side 28 intersect each other at an angle such that the pad 12 has a triangular cross section taken along a line extending between the first end 18 and the second end 20. Additionally, the back side 24 curves outwardly between the first lateral side 26 and the second lateral side 28 such that the back side 24 has a convex shape. The pad 12 may be comprised of a hypoallergenic material to inhibit an allergic reaction from occurring in the user as a result of the pad 12 contacting the user's skin 46.

A panel 30 is bonded to the pad 12 and the panel 30 extends outwardly beyond either side of the pad 12 to define a pair of wings 32 which each extends away the pad 12. The panel 30 has a primary end 34, a secondary end 36 and a first surface 38 extending between the primary end 34 and the secondary end 36, and the panel 30 is elongated between the primary end 34 and the secondary end 36. The first surface 38 is bonded to the back side 24 of the outer surface 22 of the pad 12 at a point is located adjacent to the first end 18 of the pad 12. The panel 30 is oriented to extend along an axis that is oriented perpendicular to an axis extending between the first end 18 and the second end 20 of the pad 12. The pad 12 is centrally positioned between the primary end 34 and the secondary end 36 of the panel 30 such that each of the wings 32 is defined between the pad 12 and a respective one of the primary end 34 and the secondary end 36.

A pair of adhesive members 40 is provided and each of the adhesive members 40 is bonded to a respective one of the wings 32 defined on the panel 30. In this way each of the adhesive members 40 can adhere to the user's skin 41 for retaining the pad 12 between the user's buttocks 16. Each of the adhesive members 40 has a primary surface 42 and a secondary surface 44, and the primary surface 42 of each of the adhesive members 40 is bonded to the first surface 38 of the pad 12. Furthermore, each of the adhesive members 40 is positioned on a respective one of the wings 32 defined by the panel 30 such that the secondary surface 44 of each of the adhesive members 40 adheres to the user's skin 46. Each of the adhesive members 40 may be comprised of a hypoallergenic adhesive that will not cause injury to the user's skin 41 when the adhesive members 40 are removed from the user's skin 41.

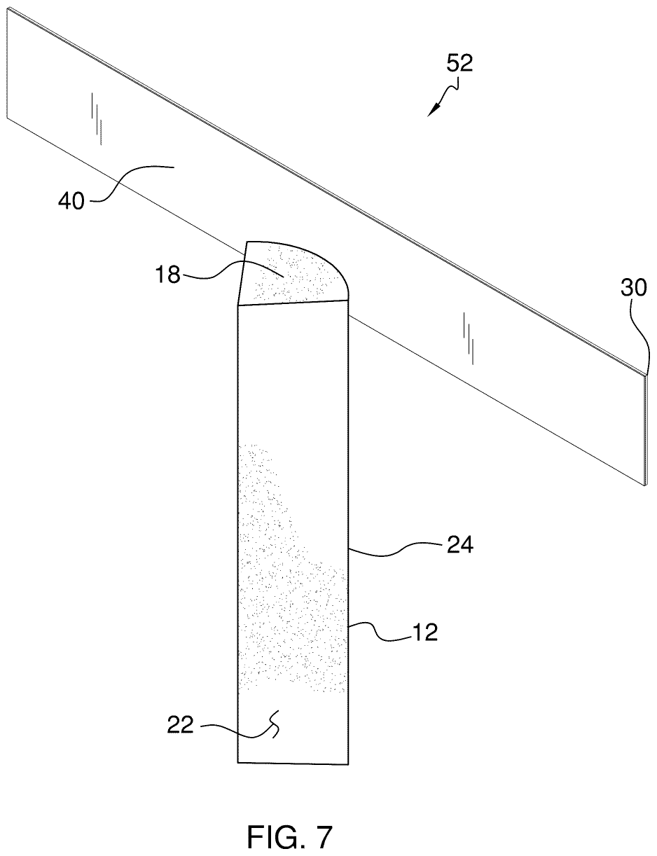

A pair of protective sheets 48 is provided and each of the protective sheets 48 is removably positioned on the secondary surface 44 of a respective one of the adhesive members 40. In this way the protective sheets 48 inhibit the secondary surface 44 from being exposed. Each of the protective sheets 48 completely covers the secondary surface 44 of the respective adhesive member 40. Additionally, each of the protective sheets 48 is removed from the respective adhesive member 40 prior to employing the pad 12. In an alternative embodiment 52 as is most clearly shown in FIG. 7, the panel 30 may be positioned on the first end 18 of the pad 12 and the panel 30 may be aligned with the back side 24 of the outer surface 22 of the pad 12. Continuing in the alternative embodiment 52, a single adhesive member 40 may be positioned on the first surface 38 of the panel 30.

In use, each of the protective sheets 48 is removed from the respective adhesive member 40 and the pad 12 is positioned between the user's buttocks 16. Each of the wings 32 is positioned to extend across the user's lower back 50 such that the adhesive members 40 adhere to the skin on the user's lower back 50. In this way the pad 12 is retained in a preferred orientation between the user's buttocks 16 such that the pad 12 can absorb menstrual fluids, for example, that might have leaked from a feminine hygiene pad. Thus, the pad 12 inhibits the user's clothing from becoming soiled by bodily fluids. The pad 12 and each of the adhesive members 40 are removed and discarded after being used.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An absorbent pad assembly for absorbing bodily fluids that leak from a feminine hygiene pad, said assembly comprising:
a pad having a plurality of intersecting sides such that said pad has a wedge shape wherein said pad is configured to be positioned between a user's buttocks, said pad being comprised of a fluid absorbent material wherein said pad is configured to absorb bodily fluids;
a panel being bonded to said pad, said panel extending outwardly beyond either side of said pad to define a pair of wings each extending away said pad; and
a pair of adhesive members, each of said adhesive members being bonded to a respective one of said wings defined on said panel wherein each of said adhesive members is configured to adhere to the user's skin for retaining said pad between the user's buttocks.

2. The assembly according to claim 1, wherein said pad has a first end, a second end and an outer surface extending between said first end and said second end, said pad being elongated between said first end and said second end, each of said intersecting sides of said pad being associated with said outer surface, said plurality of intersecting sides including a back side, a first lateral side and a second lateral side, each of said first lateral side and said second lateral side intersecting each other at an angle such that said pad has a triangular cross section taken along a line extending between said first end and said second end, said back side curving outwardly between said first lateral side and said second lateral side such that said back side has a convex shape.

3. The assembly according to claim 1, wherein said panel has a primary end, a secondary end and a first surface extending between said primary end and said secondary end, said panel being elongated between said primary end and said secondary end, said first surface being bonded to said back side of said outer surface of said pad at a point being located adjacent to said first end of said pad, said panel being oriented to extend along an axis being oriented perpendicular to an axis extending between said first end and said second end of said pad.

4. The assembly according to claim 3, wherein said pad is centrally positioned between said primary end and said secondary end of said panel such that each of said wings is defined between said pad and a respective one of said primary end and said secondary end.

5. The assembly according to claim 3, wherein each of said adhesive members has a primary surface and a secondary surface, said primary surface of each of said adhesive members being bonded to said first surface of said pad, each of said adhesive members being positioned on a respective one of said wings defined by said panel wherein said secondary surface of each of said adhesive members is configured to adhere to the user's skin.

6. The assembly according to claim 5, further comprising a pair of protective sheets, each of said protective sheets being removably positioned on said secondary surface of a respective one of said adhesive members thereby inhibiting said secondary surface from being exposed, each of said protective sheets completely covering said secondary surface of said respective adhesive member, each of said protective sheets being removed from said respective adhesive member prior to employing said pad.

7. An absorbent pad assembly for absorbing bodily fluids that leak from a feminine hygiene pad, said assembly comprising:
a pad having a plurality of intersecting sides such that said pad has a wedge shape wherein said pad is configured to be positioned between a user's buttocks, said pad being comprised of a fluid absorbent material wherein said pad is configured to absorb bodily fluids, said pad having a first end, a second end and an outer surface extending between said first end and said second end, said pad being elongated between said first end and said second end, each of said intersecting sides of said pad being associated with said outer surface, said plurality of intersecting sides including a back side, a first lateral side and a second lateral side, each of said first lateral side and said second lateral side intersecting each other at an angle such that said pad has a triangular cross section taken along a line extending between said first end and said second end, said back side curving outwardly between said first lateral side and said second lateral side such that said back side has a convex shape;
a panel being bonded to said pad, said panel extending outwardly beyond either side of said pad to define a pair of wings each extending away said pad, said panel having a primary end, a secondary end and a first surface extending between said primary end and said secondary end, said panel being elongated between said primary end and said secondary end, said first surface being bonded to said back side of said outer surface of said pad at a point being located adjacent to said first end of said pad, said panel being oriented to extend along an axis being oriented perpendicular to an axis extending between said first end and said second end of said pad, said pad being centrally positioned between said primary end and said secondary end of said panel such that each of said wings is defined between said pad and a respective one of said primary end and said secondary end;
a pair of adhesive members, each of said adhesive members being bonded to a respective one of said wings defined on said panel wherein each of said adhesive members is configured to adhere to the user's skin for retaining said pad between the user's buttocks, each of said adhesive members having a primary surface and a secondary surface, said primary surface of each of said adhesive members being bonded to said first surface of said pad, each of said adhesive members being positioned on a respective one of said wings defined by said panel wherein said secondary surface of each of said adhesive members is configured to adhere to the user's skin; and
a pair of protective sheets, each of said protective sheets being removably positioned on said secondary surface of a respective one of said adhesive members thereby inhibiting said secondary surface from being exposed, each of said protective sheets completely covering said secondary surface of said respective adhesive member, each of said protective sheets being removed from said respective adhesive member prior to employing said pad.
